(19) United States
(12) Patent Application Publication
(10) Pub. No.: US 2024/0415646 A1
(43) Pub. Date: Dec. 19, 2024

(54) CARDIAC VALVE PROSTHESIS DELIVERY SYSTEM AND METHODS OF USE

(71) Applicant: Shifamed Holdings, LLC, Campbell, CA (US)

(72) Inventors: Alice YANG, Campbell, CA (US); Ali SALAHIEH, Saratoga, CA (US); Jasper Ellington ADAMEK-BOWERS, San Francisco, CA (US); Jonathan Hwan OAKDEN, San Jose, CA (US); Jordan SKARO, San Jose, CA (US); Nicholas J SPINELLI, San Carlos, CA (US); Connor Gordon MULCAHY, San Francisco, CA (US); Troy THORNTON, San Francisco, CA (US)

(21) Appl. No.: 18/700,621

(22) PCT Filed: Oct. 14, 2022
(86) PCT No.: PCT/US2022/078128

§ 371 Date: Apr. 11, 2024

Related U.S. Application Data
(60) Provisional application No. 63/262,552, filed on Oct. 14, 2021.

(51) Int. Cl. A61F 2/24; A61M 25/01; A61M 25/06; A61M 25/09

(52) U.S. Cl. CPC A61F 2/2427; A61M 25/0155; A61M 2025/0166; A61M 25/0662; A61M 2025/09183

(57) ABSTRACT

Delivery systems, devices, and methods for delivering a valve prosthesis to a native valve annulus are provided. The valve prosthesis may be secured to the native valve via an anchor that encircles the native valve annulus and the valve prosthesis. The anchor may be deployed around the chordae tendinea and/or leaflets of the native valve. A tether may be connected to the anchor and extend outside of the patient's body to maintain communication with the anchor. After the anchor is deployed, a guidewire may be advanced through the patient's vessels parallel to the tether and through the native valve annulus. A valve delivery catheter carrying the valve prosthesis may be advanced over the guidewire and into the native valve annulus where the valve prosthesis is released and deployed. The tether may be used to position the anchor and/or valve prosthesis during one or more operations of the valve delivery process.

### CROSS REFERENCE TO RELATED APPLICATIONS

This application claims priority to U.S. Provisional Application No. 63/262,552, filed on Oct. 14, 2021, entitled “DELIVERY SYSTEM FOR CARDIAC VALVE PROSTHESIS”, the entirety of which is incorporated herein by reference for all purposes.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Blood flow between heart chambers is regulated by native valves—the mitral valve, the aortic valve, the pulmonary valve, and the tricuspid valve. Each of these valves are passive one-way valves which open and close in response to differential pressures. Patients with valvular disease have abnormal anatomy and/or function of at least one valve. For example, a valve may suffer from insufficiency, also referred to as regurgitation, when the valve does not fully close and allows blood to flow retrograde. Valve stenosis can cause a valve to fail to open properly. Other diseases may also lead to dysfunction of the valves. While medications may be used to treat the disease, in many cases the defective valve may need to be repaired or replaced at some point during the patient's lifetime. Existing valves and surgical repair and/or replacement procedures may have relatively high risks, limited lifespans, and/or be highly invasive. Some less-invasive transcatheter options are available, however these generally are limited to aortic valve procedures, are limited in patient-to-patient flexibility, and often take longer than is desirable to implant. It would therefore be desirable to provide a less invasive procedure for repair and replacement of heart valves, including the mitral valve, quicker surgical methods, and/or prosthetic valves that can accommodate a variety of individual patients.

Additionally, existing valve repair/replacement procedures are often complicated and time-consuming. Presently-available procedures often require the placement of more than one component—for example, a prosthetic valve and a mechanism to anchor it to the native anatomy. Such procedures may utilize multiple delivery catheters to carry the various components. Further, delivery of each component may require separate access sites and paths through the patient, which can be time-consuming (particularly if components are delivered sequentially), complicated, and/or dangerous. For example, it may be difficult to align components that are delivered via separate delivery systems along different paths of access to the heart. Further, some anchoring elements may require extrusion through a native valve annulus from a low-profile (e.g., elongated) delivery configuration to an expanded configuration at or near the native valve. In at least some instances, extrusion of the anchoring elements can be complicated and may not reliably deploy into the correct expanded configuration relative to the delivery device and/or the native anatomy. Proper alignment between the prosthetic valve and any anchoring or docking component is necessary to ensure proper performance of the prosthetic heart valve. Incorrect deployment may result in additional time to retract and re-deploy the anchoring element and/or prosthetic valve, more complicated anchoring procedures, and/or damage to the native tissue. It would therefore be desirable to provide quicker, less-complicated, less dangerous, and more reliably deployable valve assemblies for valvular replacement and repair.

### SUMMARY

Described herein are delivery systems and methods for delivering a valve anchor and/or a valve prosthesis to a native valve annulus. The anchor can have a spiral shape and be deployed around the chordae and/or leaflets of the native valve annulus. A tether connected to the anchor can extend to outside of the heart and/or the patient's body. The tether may remain attached to the anchor during one or more of the deployment operations of the anchor and/or the valve prosthesis. In some examples, a valve delivery catheter may be delivered over a guidewire adjacent to the tether while the tether remains attached to the anchor to deploy the valve prosthesis within the native valve annulus and the spiral-shaped anchor. In other examples, the tether may be disconnected from the anchor prior to delivering the valve delivery catheter over the guidewire. Once the valve prosthesis is deployed, the valve delivery catheter (and the tether, if not already removed) may be removed from the patient's body, leaving the valve prosthesis in within the native valve annulus and secured in place by the surrounding anchor.

According to some aspects, a method for treating a diseased native valve in a patient comprises: encircling chordae and/or leaflets of the diseased native valve in a second chamber of the patient's heart with an anchor, the anchor having a tether attached thereto extending proximally from the second chamber through a first chamber of the patient's heart; advancing a portion of a guidewire from the first chamber of the patient's heart through an annulus of the diseased native valve to the second chamber of the patient's heart; tracking a valve delivery catheter over the guidewire, the valve delivery catheter including a valve prosthesis in a compressed configuration therein; and releasing the valve prosthesis from the valve delivery catheter to expand into the annulus of the diseased native valve and within the anchor.

The method may further comprise: collecting images as the guidewire is advanced through the annulus of the diseased native valve; and deflecting a steerable catheter to steer the guidewire through an inner diameter of the anchor. Advancing the portion of the guidewire may comprise: advancing a guidewire delivery catheter through the steerable catheter, through the annulus of the diseased native valve, and through the inner diameter of the anchor; and advancing the guidewire through the guidewire delivery catheter. The method may further comprise inflating a distal portion of the guidewire delivery catheter to form a balloon to facilitate positioning and/or imaging the guidewire. Steering the guidewire may further comprise steering the guidewire through the annulus of the diseased native valve. The guidewire may have a curvature to facilitate the imaging of the guidewire. Releasing the valve prosthesis from the valve delivery catheter may comprises: partially deploying a portion of the valve prosthesis in the second chamber; pressing the partially deployed valve prosthesis against the anchor and urging the anchor toward the annulus of the diseased native valve; and fully deploying the remainder of the valve prosthesis into the annulus and/or the first chamber of the diseased native valve. The method may further comprise: disconnecting the tether from the anchor; and retracting the tether and the valve delivery catheter from the patient's body. Disconnecting and retracting the tether may be following placement of the guidewire. Disconnecting and retracting the tether may be while tracking the valve delivery catheter over the guidewire. Disconnecting and retracting the tether may be prior to release of the valve prosthesis from the valve delivery catheter. The method may further comprise delivering the valve delivery catheter and the tether within an outer sheath. Tracking the valve delivery catheter over the guidewire may further comprise running the tether through a monorail lumen of the valve delivery catheter. The method may further comprise delivering the anchor to the diseased native valve with an anchor control catheter.

According to some aspects, a delivery system for delivering a valve prosthesis to a diseased valve of a heart comprises: a tether sized and shaped to extend from outside of the heart and through an annulus of the diseased valve, the tether further configured to connect to an anchor sized and shaped to circumscribe native leaflets and/or chordae of the heart; a guidewire sized and shaped to extend from outside of the heart and through the annulus of the diseased valve; and a valve delivery catheter configured to extend over the guidewire through the annulus of the diseased valve, the valve delivery catheter configured to hold the valve prosthesis in a compressed state therein, and to release the valve prosthesis within the annulus of the diseased valve and the anchor while the tether is connected to the anchor.

The valve delivery catheter may further comprise a monorail lumen that is sized and shaped to accommodate the tether therein. The tether may further be configured for movement within the monorail lumen to adjust a position of the anchor with respect to the valve delivery catheter and/or the diseased valve. The system may further comprise a balloon catheter that is configured to extend over at least a portion of the guidewire, the balloon catheter comprising an inflatable distal portion that is sized and shaped to prevent passage thereof between adjacent chordae of the heart. The balloon catheter may be adapted for visualization via imaging. A distal portion of the guidewire may have a curvature adapted for visualization via imaging. The valve prosthesis may include an expandable frame. The tether may be releasably attached to the anchor via a releasable connector.

According to some aspects, a method of delivering a valve prosthesis in a patient's heart comprises: implanting a spiral anchor near a native valve annulus of the patient's heart by encircling chordae and/or leaflets of the native valve using an anchor control catheter, wherein a proximal end of the spiral anchor is releasably connected to a tether extending through the annulus and that is accessible outside of the patient's heart; deploying the spiral anchor from the anchor control catheter; releasing the tether from the proximal end of the spiral anchor; advancing a guidewire through the annulus of the native valve and through a central opening of the anchor; tracking a valve delivery catheter over the guidewire, the valve delivery catheter carrying the valve prosthesis in a compressed state therein; and expanding the valve prosthesis within the native valve annulus and into the central opening of the anchor.

Expanding the valve prosthesis may comprise expanding a distal portion of the valve prosthesis distally with respect to the anchor into a ventricle of the patient's heart, wherein the method may further comprise pulling the distal portion of the valve prosthesis in a proximal direction against the anchor to adjust a position of the anchor relative to the annulus of the native valve. The method may further comprise expanding a proximal portion of the valve prosthesis proximally with respect to the anchor into an atrium of the patient's heart, thereby fully deploying the valve prosthesis within the anchor and the native valve. The method may further comprise expanding a midsection of the valve prosthesis into the anchor. The method may further comprise confirming a position of the anchor relative to the annulus of the native valve prior to releasing the tether. Confirming the position of the anchor may comprise visualizing the anchor using ultrasound, fluoroscopic and/or radiographic imaging. Encircling the chordae and/or leaflets may comprise rotating the anchor control catheter with respect to the chordae and/or leaflets. The method may further comprise removing the tether from the patient's heart prior to advancing the guidewire.

According to some aspects, a method for treating a diseased native valve in a patient comprises: encircling chordae and/or leaflets of the diseased native valve with an anchor in a second chamber of the patient's heart, the anchor having a tether attached thereto, wherein the tether extends distally from a first chamber of the patient's heart, through an annulus of the diseased native valve and into the second chamber of the patient's heart; releasing the tether from the anchor once the anchor is deployed around the chordae and/or leaflets within the second chamber of the patient's heart; retracting the released tether proximally from the second chamber of the patient's heart; advancing a guidewire catheter carrying a guidewire through the first chamber of the patient's heart, through the annulus of the diseased native valve, and into the second chamber of the patient's heart; and confirming the guidewire is free of entanglement within the second chamber by advancing and retracting the guidewire catheter while visualizing a distal feature thereof.

The method may further comprise: tracking a valve delivery catheter over the guidewire, the valve delivery catheter including a valve prosthesis in a compressed configuration therein; and releasing the valve prosthesis from the valve delivery catheter to expand into the annulus of the diseased native valve and within the anchor. The guidewire may be advanced through an inner circumference of the anchor that is anchored to the diseased native valve in the second chamber of the patient's heart. Releasing the valve prosthesis from the valve delivery catheter may comprise releasing a distal portion of the valve prosthesis into the second chamber and adjusting a position of the anchor relative to the annulus of the diseased native valve using the distal portion of the valve prosthesis. The method may further comprise confirming a position of the anchor relative to the annulus of the diseased native valve by visualizing the anchor using ultrasound, fluoroscopic and/or radiographic imaging prior to releasing the tether from the anchor.

According to some aspects, a method for treating a diseased native valve in a patient comprises: advancing a guidewire through an annulus of the diseased native valve and an inner circumference of an anchor, the anchor anchored to the diseased native valve within a second chamber of the patient's heart, wherein the anchor includes a tether attached thereto extending from the second chamber through the annulus and external to the patient; tracking a valve delivery catheter over the guidewire, the valve delivery catheter including a valve prosthesis in a compressed configuration therein; and deploying the valve prosthesis from the valve delivery catheter into the annulus of the diseased native valve and within the anchor, wherein the tether is released from the anchor during deployment of the valve prosthesis.

Deploying the valve prosthesis may comprise: (a) releasing a distal portion of the valve prosthesis into the second chamber and adjusting a position of the anchor relative to the annulus of the diseased native valve using the distal portion of the valve prosthesis; and (b) releasing a proximal portion of the valve prosthesis into a first chamber of the patient's heart; wherein the tether is release from the anchor during (a), during (b), or between (a) and (b). The method may further comprise, prior to advancing the guidewire through the annulus of the diseased native valve and the inner circumference of the anchor: encircling chordae and/or leaflets of the diseased native valve with the anchor in the second chamber of the patient's heart while the tether is attached to the anchor. The tether may extend proximally from the second chamber of the patient's heart, through the annulus of the diseased native valve and into a first chamber of the patient's heart. The method may further comprise confirming a position of the anchor relative to the annulus of the diseased native valve by visualizing the anchor using ultrasound, fluoroscopic and/or radiographic imaging prior to releasing the tether from the anchor.

These and other aspects are described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1J show an exemplary method of delivering an anchor and a valve prosthesis near a native valve where a tether remains connected to the anchor while the anchor is deployed and a valve delivery catheter is deployed;

FIG. 2 shows an exemplary valve delivery catheter having a monorail lumen for a tether;

FIGS. 3A-3G show another exemplary method of delivering an anchor and a valve prosthesis near a native valve where a tether is connected to the anchor while the anchor is deployed and removed prior to deployment of a valve delivery catheter; and

FIG. 4 is a flowchart illustrating an exemplary method of deploying a prosthetic valve.

### DETAILED DESCRIPTION

Described herein are devices and methods for use in delivering a valve prosthesis, for example, during a mitral valve replacement. The delivery system may include an anchor delivery system (e.g., sub-system) to deliver a spiral shaped anchor around the patient's native valve, and a valve delivery system (e.g., sub-system) to deliver the valve prosthesis within the deployed anchor and the patient's native valve. The anchor delivery system may include an anchor control catheter for releasing and positioning the anchor within the heart. The valve delivery system may include a valve delivery catheter to position and release the valve prosthesis within the native valve annulus and a central opening of the anchor. A tether, which is connected to an end of the anchor, may be positioned in parallel to the anchor control catheter and/or valve delivery catheter. The tether may be used to adjust a position of the anchor and/or valve prosthesis, or otherwise provide access to the anchor. Such methods can advantageously allow for reliable control of the anchor and/or valve prosthesis during various operations. In some cases, the anchor delivery system and/or the valve delivery system may include one or more features to allow visualization (e.g., via ultrasound and/or fluoroscopy) to track the position of the anchor and/or the valve prosthesis within the heart.

The tether may serve one or more purposes during various operations of the anchor and/or valve delivery processes. For example, the tether may facilitate retraction of the anchor control catheter as it is being proximally pulled out of the heart. The tether may additionally or alternatively be used to adjust a position of the anchor once the anchor control catheter has been removed from the heart. This may involve pulling the tether in a proximal direction and/or pushing the tether in a distal direction to reposition the anchor close to the native valve annulus and/or to achieve a coplanar anchor orientation with respect to the native valve annulus. The tether may additionally or alternatively be used in conjunction with the valve prosthesis (e.g., before the valve prosthesis is fully deployed) to adjust a position of the anchor and the valve prosthesis.

The tether may be released from the anchor at different times of the anchor and/or valve prosthesis delivery operations. For example, the tether may be released from the anchor after the anchor has been positioned by the anchor control catheter. In other examples, the tether may be release from the anchor after a guidewire has been introduced through the anchor and/or after the valve prosthesis has been partially deployed within the heart. In further examples, the tether may be release from the anchor after the valve prosthesis has been fully deployed within the heart.

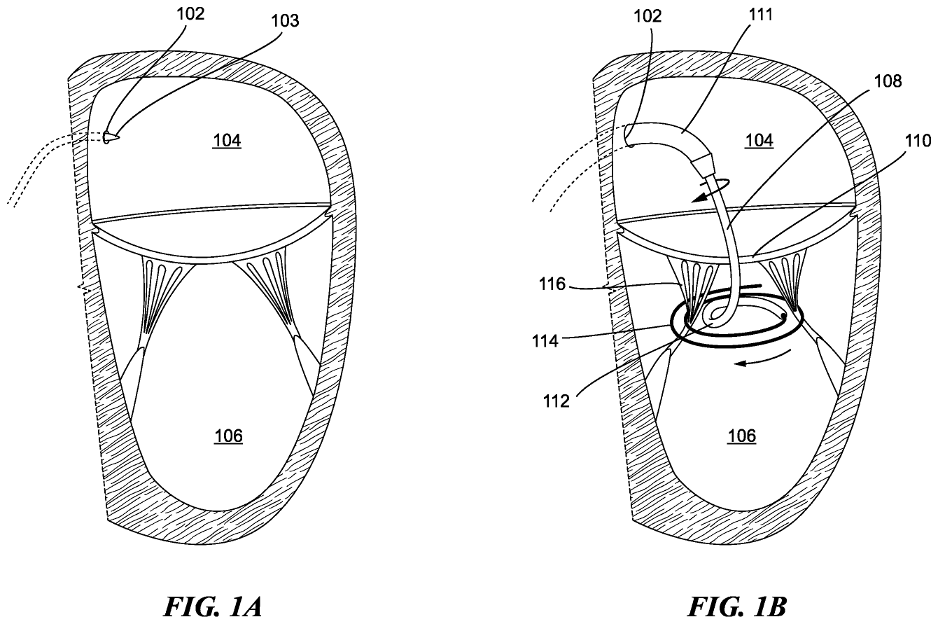

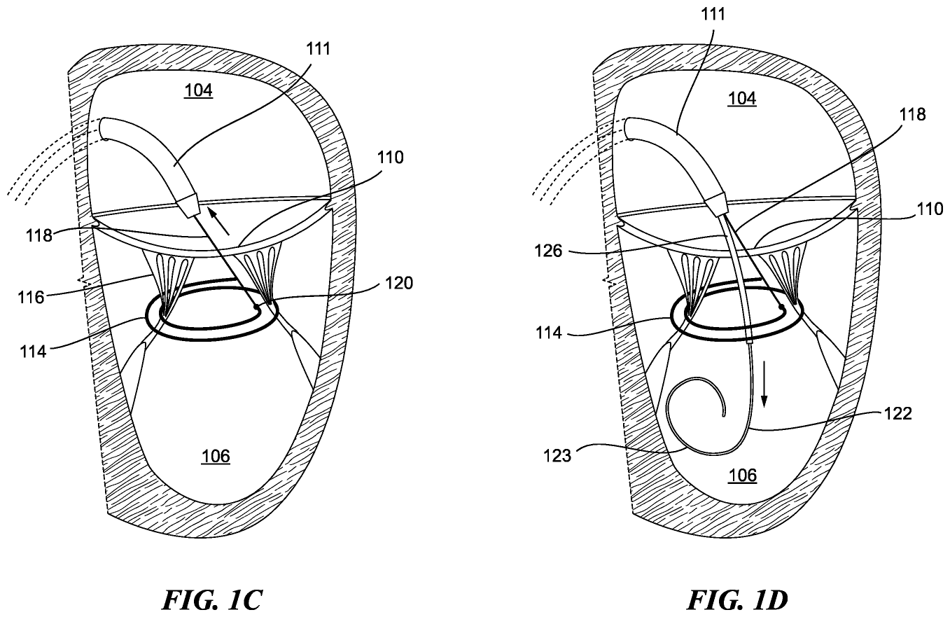

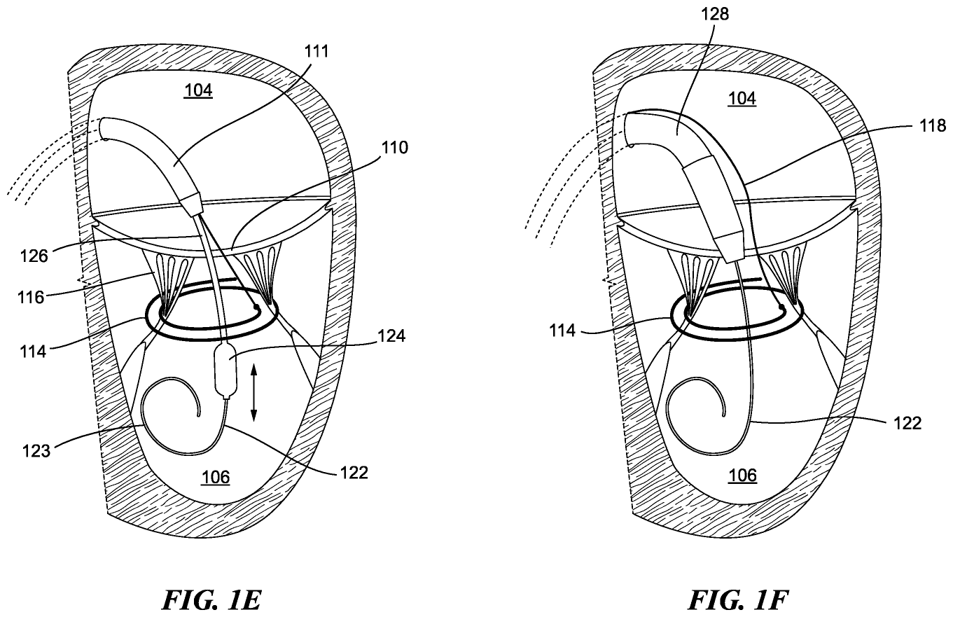

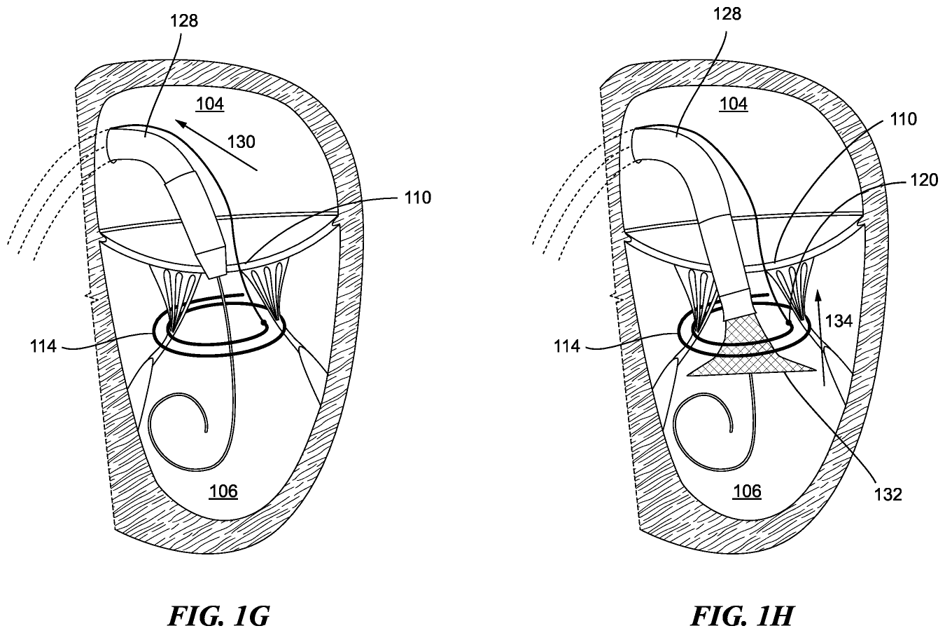

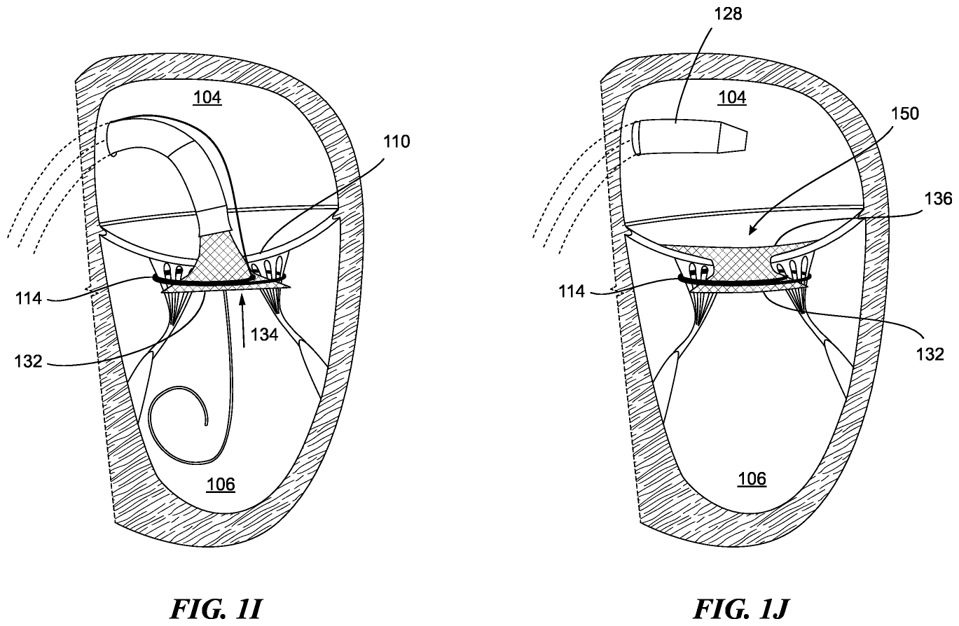

FIGS. 1A-1J show an exemplary method of delivering a prosthetic valve according to some embodiments. FIG. 1A shows a transseptal puncture 102 made by a puncture device 103 to provide access into a first chamber of the heart (in this case, the left atrium 104). The puncture device 103 may be advanced through (or be part of) steerable catheter (e.g., 111, FIG. 1B) and/or an anchor control catheter (e.g., 108, FIG. 1B), or may a separate device that is retracted prior to advancing the steerable catheter and/or anchor delivery catheter. In some cases, the puncture device 103 may include a dilator that is configured to dilate the transseptal puncture 102, for example, to accommodate entry of the anchor delivery catheter and/or valve delivery catheter.

FIG. 1B shows the steerable catheter 111 and the anchor control catheter 108 (also referred to as an anchor delivery catheter) passed through the transseptal puncture 102 and positioned for delivery of an anchor 114. The steerable catheter 111 and the anchor control catheter 108 may be part of an anchor delivery system. At least a portion of the steerable catheter 111 (e.g., distal end) may be steerable (e.g., deflectable) to steer the anchor control catheter 108 and/or the anchor 114 into a desired position and orientation relative to the native valve annulus 110. In some examples, the steerable catheter 111 and/or the anchor control catheter 108 may include one or more concentric catheters (e.g., sub-catheters) that are each configured to deflect along a plane in 3D space. Thus, a steerable catheter 111 and/or the anchor control catheter 108 with one plane-deflectable catheter may allow the steerable catheter 111 to deflect along one plane in 3D space, two concentric one plane-deflectable catheters (e.g., sub-catheters) may allow the steerable catheter 111 to deflect along two planes of 3D space, and three concentric one plane-deflectable catheters (e.g., sub-catheters) may allow the steerable catheter 111 and/or the anchor control catheter 108 to deflect along three planes of 3D space, etc. The steerable catheter 111 and/or the anchor control catheter 108 may be configured to be steerable in any number of planes and dimensions in 3D space. The steerable catheter 111 and/or anchor control catheter 108 may be bendable between a straight configuration and a bent (deflected) configuration. Such bending can be controlled, for example, at a handle operationally coupled to the steerable catheter 111 and/or anchor control catheter 108. In some cases, each inner catheter of concentric catheters may be advanceable through a distal end of a corresponding outer catheter.

Once in the first chamber of the heart (e.g., left atrium 104), the anchor control catheter 108 may be advanced through the steerable catheter 111, and the anchor 114 may be advanced through a distal guide arm portion 112 of the anchor control catheter 108. The guide arm portion 112 may be integrally formed with the rest of the anchor control catheter 108. The anchor 114 and the guide arm portion 112 of the anchor control catheter 108 may be positioned through the native valve annulus 110 and into a second chamber of the heart (in this case, the left ventricle 106). Once within the second chamber of the heart (e.g., left ventricle 106), the anchor 114 may be released from the anchor control catheter 108 and guided around the chordae tendineae 116 and/or leaflets of the of the native valve annulus 110. Engagement and positioning of the anchor 114 may involve rotating the anchor control catheter 108 (which rotates the anchor 114) with respect to chordae 116 and/or leaflets. This may involve rotating the anchor control catheter 108 with respect to the steerable catheter 111. The delivered anchor 114 may solely reside in the second chamber of the heart (e.g., left ventricle 106).

As shown, the anchor 114 may be configured to take on a spiral shape. The anchor may have one or more turns in a deployed state. In some embodiments the turns of the deployed anchor lie substantially within a (e.g., radial) plane. In some embodiments the turns of the deployed anchor at least partially overlap one another (e.g., in a direction perpendicular to the radial plane). In some embodiments the anchor is sized and shaped to be deployed in a single chamber of the heart, and to reside in a (e.g., different) single chamber of the heart. For example, the anchor can be (e.g., fully) deployed in an atrium of the heart, and implanted (e.g., solely) in a ventricle of the heart. In some examples, the anchor 114 comprises a shape memory material (e.g., nitinol, NiTi). The guide arm portion 112 may be configured to take on a pre-determined curved shape to facilitate winding of the turns of the spiral-shaped anchor 114 such that spiral arms of the anchor 114 encircle the chordae tendineae 116 and/or the leaflets. The geometry (e.g., curvature) of the guide arm portion 112 may cause torsion on the anchor 114 during deployment. In other cases, the guide arm portion 112 may be configured to take on the curved shape when activated (e.g., by a control at the handle). For example, the guide arm portion 112 may take its shape by “self-assembly” once advanced from steerable catheter 111. The curved shape of the arm portion 112 may maintain the planar orientation of the anchor 114 relative to the native valve annulus 110. The guide arm portion 112 may be positioned and/or oriented as desired by steering (e.g., deflecting, bending) the steerable catheter 111 and/or rotating the anchor control catheter 108 and/or guide arm portion 112 to encircle the chordae 116 and/or leaflets. In some embodiments, counter-rotation of the anchor 114 (via counter-rotation of the anchor control catheter 108 and/or guide arm portion 112) may aid in advancing the anchor 114 across the native valve annulus 110 without entanglement of the chordae 116 and/or leaflets. In some cases, imaging (e.g., ultrasound and/or fluoroscopy) may be used to determine whether the anchor 114 encircles enough of the chordae 116 and/or leaflets, and counter-rotating and rotating the anchor 114 as necessary to capture enough of the chordae 116 and/or leaflets.

Once the anchor 114 is at a selected depth within second chamber of the heart (e.g., the left ventricle 106), forward rotation of the anchor 114 (via forward rotation of the anchor control catheter 108 and/or guide arm portion 112) may enable the anchor 114 to encircle the mitral leaflets and chordae 116 and/or leaflets. In some embodiments, the anchor 114 is (e.g., initially) deployed towards the ventricle apex to aid in avoiding interference with leaflet motion of the native valve 110.

FIG. 1C shows the anchor 114 deployed around the chordae tendineae 116 and/or leaflets and the anchor control catheter 108 (including the guide arm portion 112) (FIG. 1B) retracted back into steerable catheter 111. As shown, a proximal end of the anchor 114 may be connected to a tether 118 that extends out of the patient's body to maintain communication with the anchor 114. A connector 120 may be adapted to releasably connect the distal end of the tether 118 to the proximal end of the anchor 114. For example, a locking mechanism of the connector 120 may be configured to lock and unlock (and disconnect) the anchor 114 to/from the tether 118 upon actuation of a handle at a proximal end of the tether 118.

In some cases, the tether 118 may be configured to take on different shapes and/or stiffnesses. For example, the tether 118 may be stiffened (and/or take on a predetermined shape) such that the tether 118 may be pushed distally (e.g., instead of or in addition to being pulled proximally) to adjust a position of the anchor 118 closer to the native valve annulus 110 and/or adjust an orientation of the anchor 118 to be coplanar with the native valve annulus 110. In some examples, the tether 118 may be stiffened (and/or take on a predetermined shape) such that the tether 118 may be pushed distally (instead of or in addition to being pulled proximally) to adjust a position of the anchor 114 closer to the native valve annulus 110 and/or an orientation of the anchor 114 with respect to the native valve annulus 110. Exemplary embodiments of suitable tethers may be described in International Application No. PCT/US2022/075931, filed on Sep. 2, 2022, the entire disclosure of which is incorporated by reference herein.

FIG. 1D shows a guidewire 122 advanced through the steerable catheter 111 adjacent to the tether 118, through the native valve annulus 110, through the inner circumference of the anchor 114, and into the second chamber of the heart (e.g., left ventricle 106). In some cases, the guidewire 112 may be delivered through a guidewire delivery catheter 126, which may itself be delivered through the steerable catheter 111. For example, the guidewire delivery catheter 126 may be advanced through the steerable catheter 111, through the native valve annulus 110 and through the inner diameter of the anchor 114; and the guidewire 123 may be advanced through the guidewire delivery catheter. In some examples, the steerable catheter 111 is steered (e.g., deflected) to position the guidewire 122 (and guidewire delivery catheter 126) through the valve annulus 110 and the inner diameter of the anchor 114. In some examples, a distal end 123 of the guidewire 122 may include a curvature (e.g., pigtail) that may facilitate the visualization of the guidewire 122 (e.g., via fluoroscopy imaging) during positioning of the guidewire 122. The guidewire 114 may be made of any of a number of materials, such one or more metals and/or one or more polymer materials. In some examples, the guidewire 122 has a diameter ranging from about 0.02 inches to about 0.05 inches (e.g., 0.02, 0.025, 0.03, 0.035, 0.04, 0.045, or 0.05 inches).

FIG. 1E shows a balloon 124 (e.g., scout balloon) that may optionally be used facilitate visualization of the guidewire 122. In some cases, the balloon 124 may be attached to or be part of the guidewire delivery catheter 126. For example, the balloon 124 may be an inflatable portion of the guidewire delivery catheter 126. The balloon 124 may be deployed (e.g., inflated) at any desirable point along the guidewire 122. For example, the balloon 124 may be at a distal end of the guidewire delivery catheter 126 and is advanced over the guidewire 122 toward the distal end 123 of the guidewire 122 where the balloon 124 may be deployed (e.g., inflated). The curvature (e.g., pigtail) of the distal end 123 of the guidewire 122 and/or the balloon 124 may be visualized (e.g., via ultrasound and/or fluoroscopy imaging) to ensure proper positioning of the guidewire 122 through substantially the center of the native valve 110, within the inner circumference of the anchor 114 and/or within the second chamber of the heart (e.g., left ventricle 106). Additionally or alternatively, the expanded balloon and visualization thereof may be used to determine undesirable placement of the guidewire 122, such as to ensure that the guidewire 122 is not tangled within or between the chordae tendineae 116 and/or leaflets, threaded through turns of the anchor 114, or not properly engage with the chordae tendineae 116 and/or leaflets. For example, the balloon can be shaped and sized so as to be prevented from passing between adjacent chordae, thus a freely moving balloon provides confirmation that the guidewire is not mispositioned between chordae or around a papillary muscle. Other imaging techniques may also be used. For example, the guidewire delivery catheter 126, the balloon 124 and/or the guidewire 122 may additionally or alternatively include one or more radio-opaque markers that are visible using radiographic imaging. Proper positioning of the guidewire 122 may ensure proper positioning of the later introduced valve delivery catheter and/or the valve prosthesis, which may track on the guidewire 122. If the guidewire 122 is found to be malpositioned, the guidewire 122 position may be adjusted. For example, the guidewire 122 may be pulled proximally, pushed distally and/or steered by activating the steerable catheter 111. After the guidewire 122 is properly positioned, the steerable catheter 111 and guidewire delivery catheter 126 may be retracted from the heart (e.g., prior to inserting a valve delivery catheter 128 as described below).

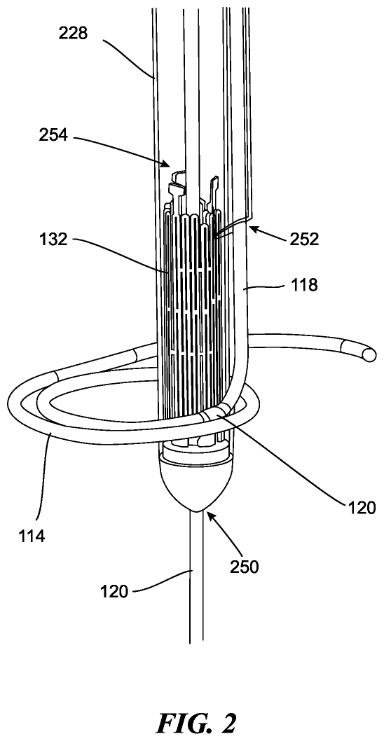

FIG. 1F shows the advancement of a valve delivery catheter 128 (as part of a valve delivery system) passed through the transseptal puncture 102 and positioned for delivery of a prosthetic valve. In the example shown, the valve delivery catheter 128 is advanced adjacent to the tether 118. The valve delivery catheter 128 may be steerable in that it may bend in any of various directions to direct a distal end of the valve delivery catheter 128 through the native valve annulus 110. For example, the valve delivery catheter 128 may be configured to bend along one or more planes in 3D space, similar to the steerable catheter 111 of the anchor delivery system. In some examples, an outer sheath (not shown) encompasses both the valve delivery catheter 128 and the tether 118. In some examples, such outer sheath may be a safety feature to minimize tissue damage at the puncture site and/or along the vessels in which the catheter(s) traverse. In some examples, the valve delivery catheter 128 may include a monorail lumen (e.g., FIG. 2) that runs parallel to a central lumen of the valve delivery catheter 128 (e.g., holding the valve prosthesis), which may be sized and shaped to accommodate the tether 118 therein. This configuration may provide more control over the tether 118 (e.g., prevent the tether from tangling).

FIG. 1G shows how tension may be placed on the tether 118 to maintain the position of the anchor 114 during advancement and manipulation of the valve delivery catheter 128. For example, it may be desirable for the anchor 114 to be maintained as close to the native valve annulus 110 as possible. Such tension may be applied by pulling the tether 118 in a proximal direction 130.

FIG. 1H shows a distal portion 132 of a valve prosthesis being deployed from the valve delivery catheter 128 into the native valve annulus 110 and the central opening of the anchor 114. In some examples, the valve prosthesis 132 has an expandable frame structure that is configured to expand when pushed out of the distal end of the valve delivery catheter 128. The valve prosthesis 132 may define a central lumen for blood to flow through and include prosthetic leaflets. FIG. 1H shows partial deployment of the valve prosthesis 132 where a distal portion of the valve prosthesis 132 is being expanded. In some examples, the partially expanded valve prosthesis may be pulled proximally 134 (e.g., by proximally pulling the valve delivery catheter 128 and/or proximally pulling the tether 118) to maintain or adjust the position of the anchor 114 and/or the distal portion 132 of the valve prosthesis closer to the native valve annulus 110, as shown in FIG. 1I. For example, the distal portion 132 of the valve prosthesis may be pressed against the anchor 114 and urge the anchor 114 toward the native valve annulus 110. FIG. 1J shows a proximal portion 136 of the valve prosthesis deployed above the native valve annulus 110 into the first chamber (e.g., left atrium 104), thereby fully deploying the valve prosthesis 150. As shown, the valve prosthesis 150 may have an hourglass shape where the distal portion 132 and the proximal portion 136 each have wider diameters than a midsection of the valve prosthesis 150. When fully deployed, the anchor 114 may surround (e.g., encompass) the midsection of the valve prosthesis 150. After the valve prosthesis 150 is fully deployed, the valve delivery catheter 128 may be pulled proximally and out of the patient's body. The anchor 114 may be disconnected from the tether 118 by unlocking the connector 120. The tether 118 may then be removed by pulling the tether 118 proximally and out of the patient's body.

Exemplary embodiments of valve prostheses/anchors are described in U.S. Patent Publication No. US2020/0297491A1, published on Sep. 24, 2020, and U.S. Pat. No. 10,912,644, issued on Feb. 9, 2021, the entire disclosures of which are incorporated by reference herein.

FIG. 2 shows an exemplary valve delivery catheter 228 that is a variation of the valve delivery catheter 128 shown in FIGS. 1F-1J. In this variation, the valve delivery catheter 228 includes a monorail lumen 252 that is sized and shaped to accommodate the tether 118. As shown, the monorail lumen 252 may run along one side of the valve delivery catheter 228 parallel to a central lumen 250 that accommodates the guidewire 122. The monorail lumen 252 may constrain movement of the tether 118, for example, to prevent the tether 118 from twisting or becoming entangled. The central lumen 250 may define a distal opening where the guidewire 122 may exit the valve delivery catheter 228. The valve delivery catheter 228 includes an inner volume 254 that is adapted (e.g., sized and shaped) to hold the prosthetic valve 132 in a compressed configuration (e.g., prior to release and expansion of the prosthetic valve 132). The central lumen 250 may run through the center of the valve delivery catheter 228 and the prosthetic valve 132 when the prosthetic valve 132 disposed within the valve delivery catheter 228.

Examples of valve delivery catheters with monorail lumens are described in International Patent Application No. PCT/US2021/026463, filed on Apr. 8, 2021, published as WO 2021/207545 on Oct. 14, 2021, the entire disclosure of which is incorporated by reference herein.

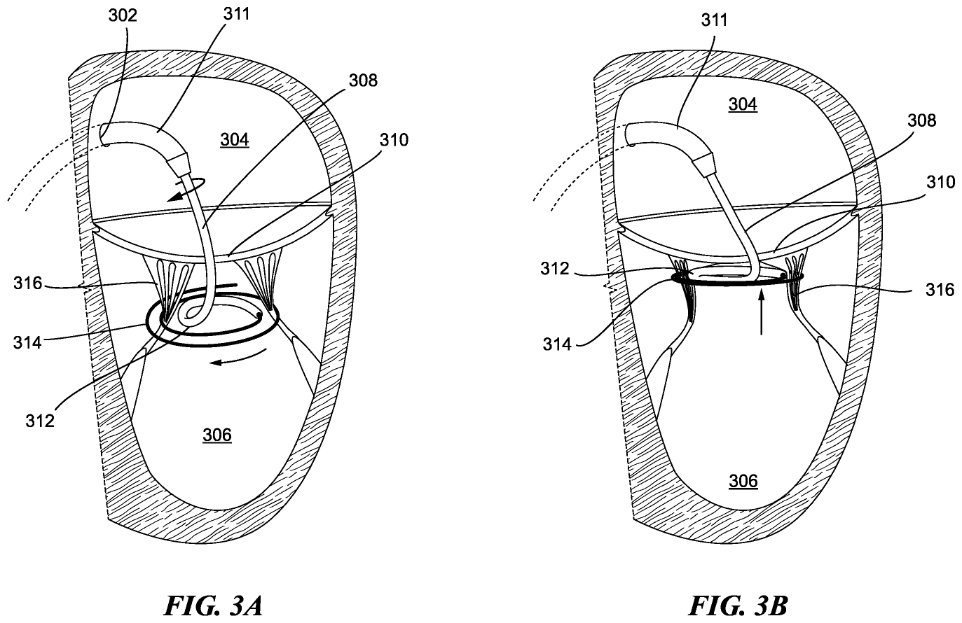

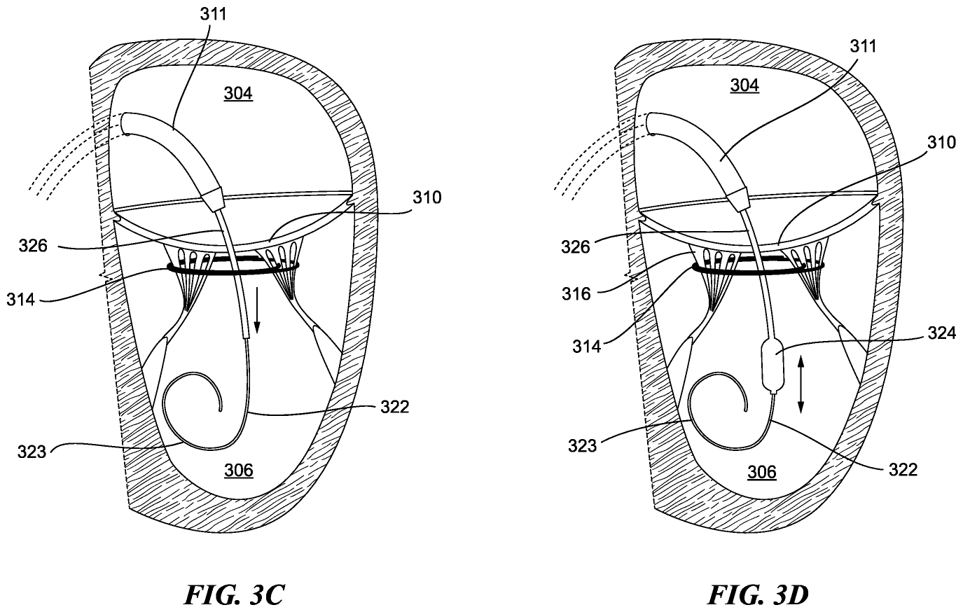

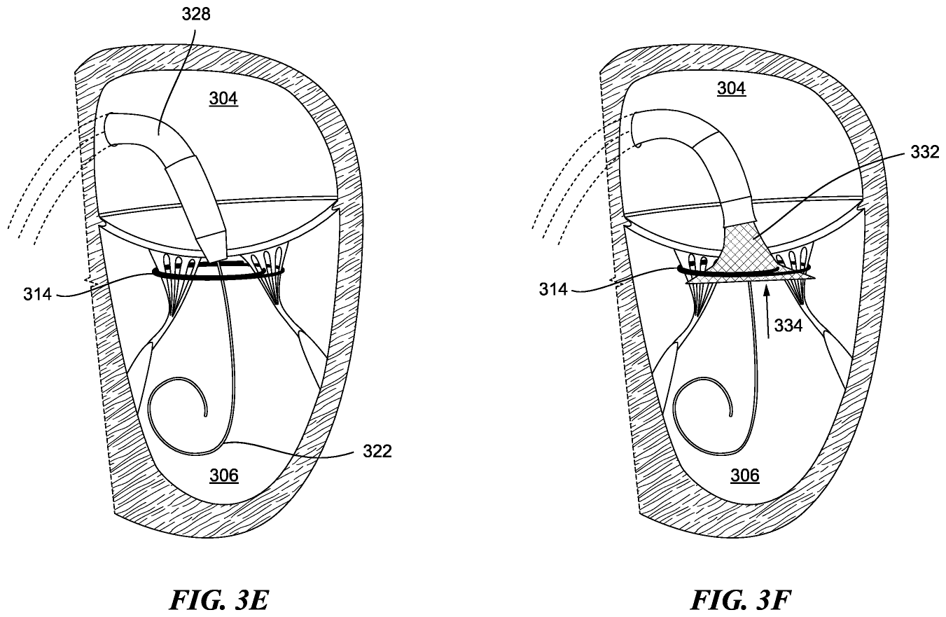

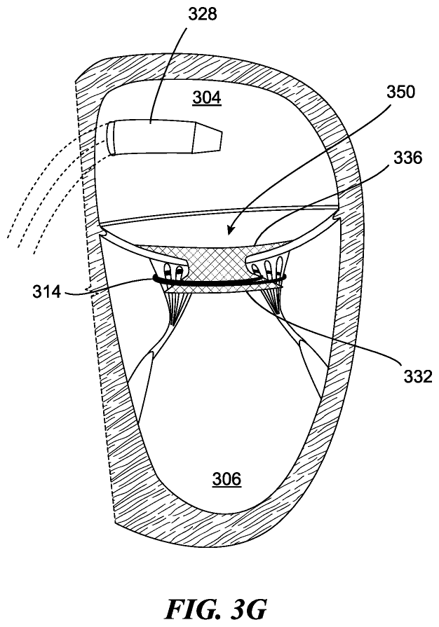

FIGS. 3A-3G show another exemplary method of delivering a prosthetic valve, in this case. The operations illustrated in FIGS. 3A-3G are similar to those in FIGS. 1B-1J except that a tether is connected during deployment of an anchor 314 but removed prior to deployment of a valve delivery catheter. FIG. 3A shows a steerable catheter 311 and an anchor control catheter 308 passed through a transseptal puncture 302 and positioned for delivery of an anchor 314. Once in a first chamber of the heart (e.g., the left atrium 304), the anchor control catheter 308 may be advanced through the steerable catheter 311, then the anchor 314 may be advanced through the anchor control catheter 308 and out of a distal guide arm portion of the 312 of the anchor control catheter 308. The anchor 314 and the guide arm portion 312 may be positioned through a native valve annulus 310 and into a second chamber of the heart (e.g., the left ventricle 306). Once within the second chamber of the heart (e.g., the left ventricle 306), the anchor 314 may be guided around the chordae tendineae 316 and/or leaflets of the of the native valve annulus 310. The anchor 314 may be positioned and/or oriented as desired by steering (e.g., deflecting, bending) the steerable catheter 311 and/or the anchor control catheter 308 (e.g., guide arm portion 312), such as described above with respect to FIG. 1B. In FIG. 3A (similar to FIG. 1B) a tether may be connected to the anchor 314 by a connector within the anchor control catheter 308.

FIG. 3B shows a position of the anchor 314 closer to and oriented in a planar configuration with respect to a plane of the native valve annulus 310. The distal guide arm portion 312 of the anchor control catheter 308 may take on the curved shape to maintain the planar orientation of the anchor 314 relative to the native valve annulus 310. In some cases, the anchor control catheter 308 transition from a straight shape when in the steerable catheter 311 to the curved shape when it is released from the steerable catheter 311. For example, the anchor control catheter 308 may be made of a shape memory material (e.g., nitinol) that is pre-treated to take on the curved shape. Alternatively or additionally, the anchor control catheter 308 may be configured to take on the curved shape upon activation (e.g., by a control of the handle). In some cases, the tether (e.g., 118), which may at least partially be within the anchor control catheter 308, may be pulled proximally (and/or pushed distally) to provide traction against the anchor 314 and change a shape of the anchor control catheter 308. This traction may keep the anchor in place as the anchor control catheter 308 is retracted proximally back through the steerable catheter 311. In some examples, imaging (e.g., ultrasound and/or fluoroscopy) may be used to visualize the anchor 314 during or after deployment of the anchor 314. Such imaging may be used to confirm proper placement and/or orientation of the anchor 314. Once the anchor 314 is properly positioned and oriented, the tether may be released from the anchor 314 by releasing a releasable connector (e.g., 120).

FIG. 3C shows a guidewire delivery catheter 326 with a guidewire 322 advanced through the steerable catheter 311, the native valve annulus 310, the inner opening of the anchor 314, and into the second chamber of the heart (e.g., left ventricle 306), similar to described previously with reference to FIG. 1D. FIG. 3D shows an optional balloon 324 (e.g., attached to or part of the guidewire delivery catheter 326) that may optionally be used facilitate visualization of the guidewire 322, similar to described previously with reference to FIG. 1E. FIG. 3E shows the advancement of a valve delivery catheter 328 (e.g., as part of a valve delivery system) passed through the transseptal puncture 302 and positioned for delivery of a prosthetic valve, similar to described previously with reference to FIG. 1F.

FIG. 3F shows a distal portion 332 of a valve prosthesis being deployed from the valve delivery catheter 328 into the native valve annulus 310 and the central opening of the anchor 314, similar to described previously with reference to FIG. 1H. In some examples, the distal portion 332 of the valve prosthesis may be pulled proximally 334 (e.g., by proximally pulling the valve delivery catheter 328) to maintain or move the position of the anchor 314 close to the native valve annulus 310, if needed. FIG. 3G shows a proximal portion 336 of the valve prosthesis deployed above the native valve annulus 310, thereby fully deploying the valve prosthesis 350. After the valve prosthesis 350 is fully deployed, the valve delivery catheter 328 may be pulled proximally and out of the patient's body.

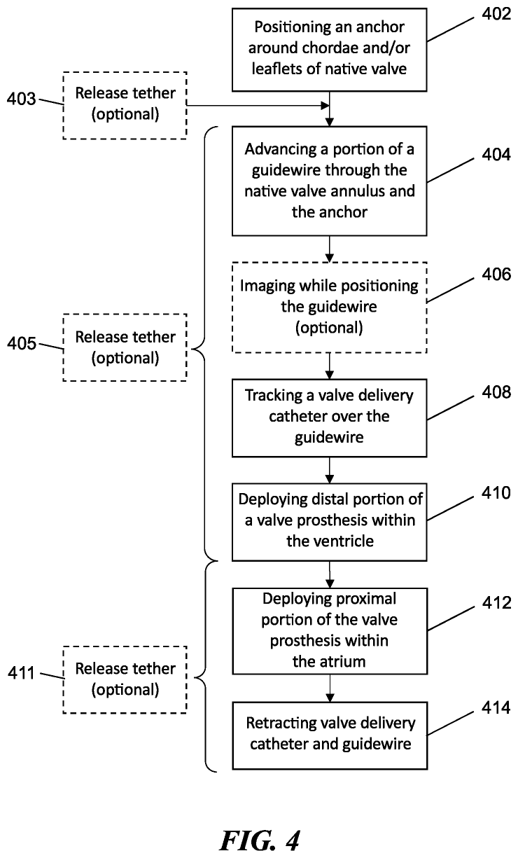

FIG. 4 is a flowchart illustrating an exemplary method of implanting a valve prosthesis for treating a diseased valve. An example operation 402 comprises positioning an anchor around chordae and/or leaflets of the diseased native valve. This can involve encircling a spiral-shaped anchor around the chordae and/or leaflets. In some examples, the anchor is delivered to the heart via a steerable catheter and/or an anchor control catheter. A guide arm portion of the anchor control catheter may extend from the steerable catheter and have a curved shape designed to guide the anchor around the chordae and/or leaflets as the anchor is advanced out of the anchor guide. The steerable catheter may be steerable (e.g., deflectable) to steer the anchor control catheter and/or the anchor into position. Additionally or alternatively, the anchor control catheter may be steerable (e.g., deflectable) to steer the anchor into position. The guide arm portion may be rotatable relative to the steerable catheter to facilitate positioning of the anchor. A tether may be attached to the anchor during deployment of the anchor around the chordae/leaflets. In some examples, the tether may be pulled proximally to provide traction against the anchor. This traction may change a shape (e.g., compress the curvature) of the anchor control catheter and/or keep the anchor in place as the anchor control catheter is retracted proximally. In some cases, imaging (e.g., ultrasound and/or fluoroscopy) may be used to visualize the anchor during or after deployment and/or to confirm proper placement and/or orientation of the anchor.

Once the anchor is deployed, at operation 403 the tether is optionally released from the anchor. For example, once the tether has been used to apply tension on the anchor control catheter and/or keep the anchor in place as the anchor control catheter is retracted proximally, the tether may be disconnected from the anchor and pulled proximally out of the patient's body. In some cases, releasing the tether from the anchor early (e.g., prior to operation 404) may prevent the tether from interfering with the position of the anchor. Further, the access to the anchor via the tether may not be necessary once the position and/or orientation of the anchor is properly positioned at operation 402.

An example operation 404 comprises advancing at least a portion of a guidewire through the annulus of the diseased native valve and the central opening of the spiral-shaped anchor. The guidewire may be passed from a first heart chamber (e.g., left atrium) to a second heart chamber (e.g., left ventricle). In some cases, a steerable catheter may be steered (e.g., deflected) to steer the guidewire through the diseased native valve annulus and the anchor. An example operation 406 comprises optionally using imaging (e.g., ultrasound and/or fluoroscopy) while positioning the guidewire through the diseased native valve annulus and the anchor. In some examples, the guidewire may have a distinctive shape (e.g., pigtail end) to facilitate identification of the guidewire and its location relative to the anchor and/or the native valve annulus using the imaging technique(s). Additionally or alternatively, the guidewire may include a balloon to facilitate identification of the guidewire and its location. Imaging may be done in real time during the placement of the guidewire to prevent the guidewire from becoming entangled within the chordae and/or leaflets, run through turns of the anchor, or become positioned outside of the chordae and/or leaflets. The steerable catheter may be steered to control the movement of the guidewire as it is extended from the steerable catheter. In some cases, the guidewire is manipulated by a combination of advancing the guidewire and retracting the guidewire relative to the steerable catheter. The imaging may also be used to confirm proper placement of the guidewire once placed through the valve annulus. Once the guidewire is properly positioned, the steerable catheter may be retracted.

An example operation 408 comprises tracking a valve delivery catheter over the guidewire. In some examples, the tether may remain attached to the anchor such that the valve delivery catheter runs parallel with the tether along a least a portion of the patient's blood vessels and through the transeptal puncture. In cases where the tether is still attached to the anchor, the valve delivery catheter and the tether may be within an outer sheath of the valve delivery catheter. The outer sheath may minimize tissue damage at the puncture site and/or along the vessels in which the valve delivery catheter (and the tether) traverse. In some examples, the valve delivery catheter is steerable. In some examples, the valve delivery catheter may include a monorail lumen to accommodate the tether therein (in cases where the tether is still present). This may control the position and movement of the tether, e.g., may prevent the tether from tangling.

An example operation 410 comprises deploying a distal portion of a valve prosthesis from the valve delivery catheter into the second side (e.g., ventricle side) of the native valve annulus. Release of the valve prosthesis from the valve delivery catheter may be controlled such that the distal portion of the valve prosthesis expands into the second side (e.g., ventricular side) of the native valve without releasing/expanding a proximal portion of the valve prosthesis within the atrial side of the native valve annulus. In some examples, the distal portion of the valve prosthesis is pulled proximally against the anchor to position the anchor and the distal portion of the valve prosthesis as close to a plane of the native valve annulus as possible and/or to achieve a coplanar orientation with respect to the plane of the native valve annulus. Once the anchor and the distal portion of the valve prosthesis is properly positioned with respect to the native valve annulus, a proximal portion of the valve prosthesis may be deployed, thereby fully deploying the valve prosthesis.

In cases where the tether has not already been released from the anchor at operation 403, at operation 405 the tether may optionally be disconnected and released from the anchor at (or between) any of the operations 404 to 410. For example, the tether may be pulled proximally and/or pushed distally at (or between) any of the operations 404 to 410 to reposition the anchor as close to the native valve annulus as possible and/or to achieve a coplanar anchor orientation with respect to the native valve annulus. In some cases, releasing the tether from the anchor at or between operations 404 to 410 may prevent the tether from interfering with the position of the anchor during these operations. Further, the access to the anchor via the tether may not be necessary after one of the operations 404 to 410.

At operation 412, a proximal portion of the valve prosthesis is deployed within the first heart chamber (e.g., atrium at an atrial side of the native valve annulus), thereby fully deploying the valve prosthesis. At operation 414, the valve delivery catheter and the guidewire may be retracted and removed from the patient's body. In cases where the tether has not already been released from the anchor at 403 or 405, the tether may be the tether may be disconnected and released from the anchor after operation 410, at (or between) any of the operations 412 to 414, or after operation 414.

Any of the methods described herein may include visualizing the anchor, the valve prosthesis, the tether and/or any of the catheters (e.g., steerable catheter, anchor control catheter, valve delivery catheter, and/or outer sheath) while in the patient's heart. The visualizing may include using one or more imaging techniques, such as (but not limited to) ultrasound, fluoroscopy and/or radiofrequency imaging techniques. The imaging may be used to visualize a position of the anchor, the valve prosthesis, the tether and/or any of the catheters at or between any of the operations 402 to 414.

As described herein, any of the valve prostheses may include a frame structure. The frame structure may be configured like a stent. The frame structure may, for example, comprise a scaffold in a diamond pattern formed from a shape memory material (e.g., nitinol, NiTi). One of ordinary skill in the art will appreciate that many other structures, materials, and configurations may be employed for the frame structure. For example, the frame structure may be formed of a polymer of sufficient elasticity. The frame structure may be formed of a combination of metal and polymer, such as metal (e.g., shape memory material) covered in polymer. The frame structure may include a variety of patterns besides diamond shapes. In some embodiments, the frame structure is a closed frame such that blood flow is forced through valve segment therein. One or more skirts and/or seals may help force blood through the valve segment.

One of ordinary skill in the art will recognize based on the description herein that any of the valve prostheses described herein may comprise any of the frame structure shapes, frame structure designs, frame structure materials, anchor shapes, anchor windings, anchor materials, free end tips, leaflet(s) configurations, or any other of the variable features described herein in any combination thereof as desired.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected,” “attached” or “coupled” to another feature or element, it can be directly connected, attached or coupled to the other feature or element or intervening features or elements may be present. In contrast, when a feature or element is referred to as being “directly connected,” “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise,” and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), or +/−10% of the stated value (or range of values). Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that these data represent endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others.

Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method for treating a diseased native valve in a patient, the method comprising:
encircling chordae and/or leaflets of the diseased native valve in a second chamber of the patient's heart with an anchor, the anchor having a tether attached thereto extending proximally from the second chamber through a first chamber of the patient's heart;
advancing a portion of a guidewire from the first chamber of the patient's heart through an annulus of the diseased native valve to the second chamber of the patient's heart;
tracking a valve delivery catheter over the guidewire, the valve delivery catheter including a valve prosthesis in a compressed configuration therein; and
releasing the valve prosthesis from the valve delivery catheter to expand into the annulus of the diseased native valve and within the anchor.

2. The method of claim 1, further comprising:
collecting images as the guidewire is advanced through the annulus of the diseased native valve; and
deflecting a steerable catheter to steer the guidewire through an inner diameter of the anchor.

3. The method of claim 2, wherein advancing the portion of the guidewire comprises:
advancing a guidewire delivery catheter through the steerable catheter, through the annulus of the diseased native valve, and through the inner diameter of the anchor; and
advancing the guidewire through the guidewire delivery catheter.

4. The method of claim 3, further comprising inflating a distal portion of the guidewire delivery catheter to form a balloon to facilitate positioning and/or imaging the guidewire.

5. The method of claim 2, wherein steering the guidewire further comprises steering the guidewire through the annulus of the diseased native valve.

6. The method of claim 2, wherein the guidewire has a curvature to facilitate the imaging of the guidewire.

7. The method of claim 1, wherein releasing the valve prosthesis from the valve delivery catheter comprises:
partially deploying a portion of the valve prosthesis in the second chamber;
pressing the partially deployed valve prosthesis against the anchor and urging the anchor toward the annulus of the diseased native valve; and
fully deploying the remainder of the valve prosthesis into the annulus and/or the first chamber of the diseased native valve.

8. The method of claim 1, further comprising:
disconnecting the tether from the anchor; and
retracting the tether and the valve delivery catheter from the patient's body.

9. The method of claim 8, wherein disconnecting and retracting the tether is following placement of the guidewire.

10. The method of claim 8, wherein disconnecting and retracting the tether is while tracking the valve delivery catheter over the guidewire.

11. The method of claim 8, wherein disconnecting and retracting the tether is prior to release of the valve prosthesis from the valve delivery catheter.

12. The method of claim 1, further comprising delivering the valve delivery catheter and the tether within an outer sheath.

13. The method of claim 1, wherein tracking the valve delivery catheter over the guidewire further comprises running the tether through a monorail lumen of the valve delivery catheter.

14. The method of claim 1, further comprising delivering the anchor to the diseased native valve with an anchor control catheter.

15. A delivery system for delivering a valve prosthesis to a diseased valve of a heart, the delivery system comprising:
a tether sized and shaped to extend from outside of the heart and through an annulus of the diseased valve, the tether further configured to connect to an anchor sized and shaped to circumscribe native leaflets and/or chordae of the heart;
a guidewire sized and shaped to extend from outside of the heart and through the annulus of the diseased valve; and
a valve delivery catheter configured to extend over the guidewire through the annulus of the diseased valve, the valve delivery catheter configured to hold the valve prosthesis in a compressed state therein, and to release the valve prosthesis within the annulus of the diseased valve and the anchor while the tether is connected to the anchor.

16. The system of claim 15, wherein the valve delivery catheter further comprises a monorail lumen that is sized and shaped to accommodate the tether therein.

17. The system of claim 16, wherein the tether is further configured for movement within the monorail lumen to adjust a position of the anchor with respect to the valve delivery catheter and/or the diseased valve.

18. The system of claim 15, further comprising a balloon catheter that is configured to extend over at least a portion of the guidewire, the balloon catheter comprising an inflatable distal portion that is sized and shaped to prevent passage thereof between adjacent chordae of the heart.

19. The system of claim 18, wherein the balloon catheter is adapted for visualization via imaging.

20. The system of claim 15, wherein a distal portion of the guidewire has a curvature adapted for visualization via imaging.

21. The system of claim 15, wherein the valve prosthesis includes an expandable frame.

22. The system of claim 15, wherein the tether is releasably attached to the anchor via a releasable connector.

23-40. (canceled)
